(19) United States
(12) Patent Application Publication
(10) Pub. No.: US 2024/0079119 A1
(43) Pub. Date: Mar. 7, 2024

(54) SYSTEMS AND METHODS FOR MEDICAL PATIENT CLASSIFICATION

(71) Applicant: MCKINSEY & COMPANY, INC., New York, NY (US)

(72) Inventors: Omar Kattan, Newport Beach, CA (US); Beth Kenworthy, Chicago, IL (US); Dina Marie Pitta, New York, NY (US); Jordan Vanlare, New Rochelle, NY (US); Anne Koffel, Boston, MA (US)

(21) Appl. No.: 18/241,511

(22) Filed: Sep. 1, 2023

Related U.S. Application Data
(60) Provisional application No. 63/403,127, filed on Sep. 1, 2022.

(51) Int. Cl. G16H 40/20; G16H 10/60; G16H 50/20

(52) U.S. Cl. CPC G16H 40/20; G16H 10/60; G16H 50/20

(57) ABSTRACT

The systems and methods disclosed herein relate generally to making organizational predictions (e.g., a prediction of an optimal staffing level), and clinical operational predictions (e.g., a prediction of an improvement to a care pathway) in a healthcare context. To this end, in some embodiments, patients are classified into hierarchical categories (e.g., categories ranging from most severe to least severe). The classifications of the patients may then be used to make the organizational prediction, and/or the clinical operational prediction.

### CROSS REFERENCE TO RELATED APPLICATIONS

This application claims the benefit of U.S. Provisional Application No. 63/403,127, entitled “Systems and Methods for Medical Patient Classification” (filed Sep. 1, 2022), the entirety of which is incorporated by reference herein.

### BACKGROUND

Healthcare providers, such as hospitals, currently face many challenges. For example, it may be difficult for a healthcare provider to determine optimal staffing levels for different types of caregivers (e.g., primary care physicians (PCPs), advanced practice providers (APPs), etc.). In another example, it may be difficult for a healthcare provider to determine what schedule of care should be provided to a patient.

The systems and methods disclosed herein provide solutions to these problems and others.

### SUMMARY

The following relates generally to predicting organizational or clinical operational improvements in a healthcare context. More specifically, the following classifies patients into categories, and then uses the classifications to predict organizational or clinical operational improvements.

In one aspect, a computer-implemented method for classifying medical patients may be provided. The method may include: (1) receiving, via one or more processors, respective medical data of a plurality of patients; (2) classifying, via the one or more processors, one or more patients of the plurality of patients into respective categories of a plurality of hierarchical categories based on the received respective medical data; (3) predicting, via the one or more processors, based on the respective classifications of the patients into the respective categories, an organizational or clinical operational improvement; and (4) displaying, on a display, the predicted organizational or clinical operational improvement.

In another aspect, a computer system for classifying medical patients may be provided. The computer system may include one or more processors configured to: (1) receive respective medical data of a plurality of patients; (2) classify one or more patients of the plurality of patients into respective categories of a plurality of hierarchical categories based on the received respective medical data; (3) predict, based on the respective classifications of the patients into the respective categories, an organizational or clinical operational improvement; and (4) display, on a display, the predicted organizational or clinical operational improvement.

In yet another aspect, a computing device for classifying medical patients may be provided. The computing device may include.

### BRIEF DESCRIPTION OF THE DRAWINGS

FIG. 1 depicts an example system for classifying medical patients, and making an organizational prediction or a clinical operational prediction, according to some aspects.

FIG. 2 depicts example classification categories, according to some aspects.

FIG. 3 depicts an example of spending based on category, according to some aspects.

FIG. 4 depicts example bundles for example categories, according to some aspects.

FIG. 5 depicts an example pathway of a patient in an unplanned inpatient (IP) admission, according to some aspects.

FIG. 6 depicts an example method of classifying medical patients, according to some aspects.

FIG. 7 depicts example categories and example criteria, according to some aspects.

Advantages will become more apparent to those skilled in the art from the following description of the preferred embodiments which have been shown and described by way of illustration. As will be realized, the present embodiments may be capable of other and different embodiments, and their details are capable of modification in various respects. Accordingly, the drawings and description are to be regarded as illustrative in nature and not as restrictive.

### DETAILED DESCRIPTION

The present embodiments relate generally to classifying patients into categories, and then using the classifications to predict organizational or clinical operational improvements. For example, a medical patient may be classified into a hierarchical category, where the hierarchical categories range from most severe (e.g., possibly requiring the most amount of care) to least severe (e.g., possibly requiring the least amount of care). The numbers of patients in each category for a healthcare facility may then be used to predict an organizational improvement (e.g., predict an optimal overall staffing level of the healthcare facility, and/or an optimal staffing level of a particular type of caregiver). Additionally or alternatively, the classifications may be used to predict a clinical operational improvement (e.g., predict an optimal bundle of services to a patient based on the patient's classification).

It should be understood that an “optimal” level is a best level based on suitable factors. For example, an optimal staffing level may be a best staffing level based on patient outcome and/or experience, financial expenditure of the patient and/or healthcare facility, etc.

### Example System

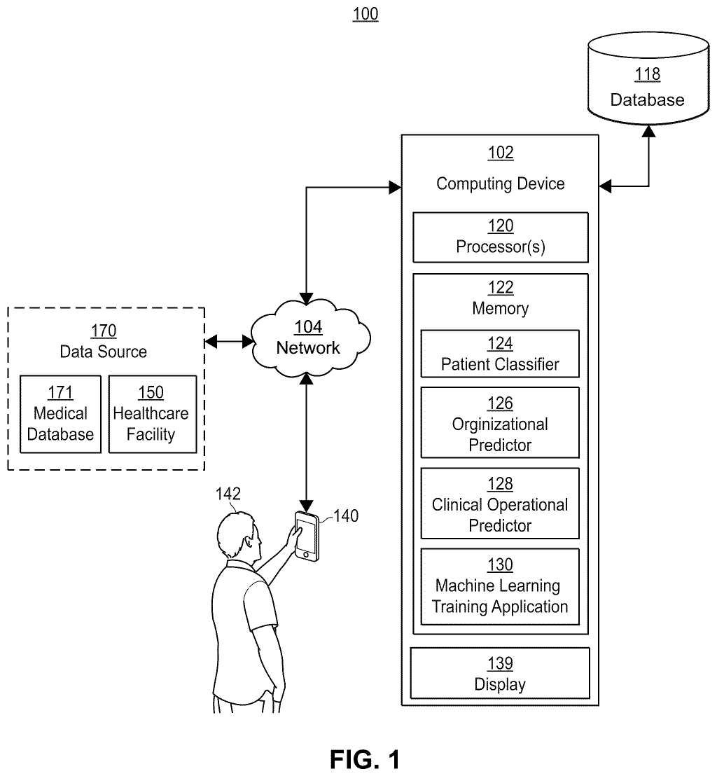

FIG. 1 shows an example system 100 for classifying medical patients. The high-level architecture illustrated in FIG. 1 may include both hardware and software applications, as well as various data communications channels for communicating data between the various hardware and software components, as is described below. The system may include a computing device 102 configured to communicate (e.g., via a network 104, which may be a wired or wireless network, such as the Internet), with a user computing device 140, and/or data source 170.

The computing device 102 may include one or more processors 120 such as one or more microprocessors, controllers, and/or any other suitable type of processor (e.g., one or more central processing units (CPUs), one or more graphics processing units (GPUs), etc.). The computing device 102 may further include a memory 122 (e.g., volatile memory, non-volatile memory) accessible by the one or more first processors 120 (e.g., via a memory controller). Additionally, the computing device 102 may include a display 139. In some embodiments, the computing device 102 is part of a cloud computing platform.

The one or more processors 120 may interact with the memory 122 to read and to execute computer-readable instructions stored in the memory 122. Additionally or alternatively, computer-readable instructions may be stored on one or more removable media (e.g., a compact disc, a digital versatile disc, removable flash memory, etc.) that may be coupled to the computing device 102 to provide access to the computer-readable instructions stored thereon. In particular, the computer-readable instructions stored on the memory 122 may include one or more sets of instructions, such as a patient classifier 124, an organizational predictor 126, a clinical operational predictor 128, and/or a machine learning training application 130. More or fewer sets of instructions may be included, in some aspects.

The computing device 102 may be any suitable device. For example, the computing device 102 may be one or more servers, one or more personal computers, one or more smartphones, one or more tablets, one or more phablets, etc.

The patient classifier 124 may be a set of computer-executable instructions accessible by the one or more processors 120. The patient classifier 124 may include computer-executable instructions that classify a patient into a hierarchical category. For instance, as will be described in further detail below, the patient classifier 124 may classify patients into hierarchical categories of increasing severity based on medical data of the patient.

The organizational predictor 126 may be a set of computer-executable instructions accessible by the one or more processors 120. The organizational predictor 126 may include computer-executable instructions that use the classifications of the patients made by the patient classifier 124 to predict an organizational improvement, such as an optimal overall staffing level of a healthcare facility, an optimal staffing level for a particular type of caregiver, an optimal ratio of a first type of caregiver to a second type of caregiver, or an amount of financing that will be required for a particular category.

The clinical operational predictor 128 may be a set of computer-executable instructions accessible by the one or more processors 120. The clinical operational predictor 128 may include computer-executable instructions that use the classifications of the patients made by the patient classifier 124 to predict a clinical operational improvement, such as an optimal bundle of services for a patient, or an improvement in a care pathway.

The machine learning training application 130 may be a set of computer-executable instructions accessible by the one or more processors 120. The machine learning training application 130 may include computer-executable instructions that train machine learning algorithm(s) that are used by other components of the computing device 102. In one example, the instructions may train a machine learning algorithm to classify subjects into categories (e.g., a machine learning algorithm used by the subject classifier 124). In another example, the instructions may train a machine learning algorithm to predict organizational improvements (e.g., a machine learning algorithm used by the organizational predictor 126). In yet another example, the instructions may train a machine learning algorithm to predict clinical operational improvements (e.g., a machine learning algorithm used by the clinical operational predictor 128).

In some aspects, the computing device 102 classifies patients into hierarchical categories based on medical data. In some embodiments, the hierarchical categories range from a most severe category (e.g., an end of life (EOL) category) to a least severe category (e.g., a wellbeing category).

The medical data that the patients are classified based on may come from any suitable source. For example, the medical data may be sent to the computing device 102 from a data source 170. One example of the data source 170 includes a healthcare facility 150, such as hospital, primary care physician (PCP), medical lab, and/or hospice facility. Another example of the data source 170 includes a medical database 171, such as a database of a medical insurance provider an electronic medical records database, or a data aggregator.

Furthermore, it should be understood that although the example of FIG. 1 illustrates only one of each of the data sources 170, any number of data sources may be used (e.g., more than one healthcare facility 150, more than one medical database 171, etc.).

The patient classifier 124 also classifies the patients based on medical data held by the database 118. An example of the database 118 includes a proprietary database owned by a company that also owns the computing device 102. Furthermore, it should be understood that although the example of FIG. 1 illustrates only one database 118, any number of databases 118 may be used.

In addition, medical data that is used to classify the patients may also be received from the user computing device 140 (e.g., a computing device, such as a smart phone, a personal computing device, a tablet, a phablet, a smart watch, a smart medical device, a medical monitor, etc.). In some examples, the user computing device 140 belongs to a user 142; for example, a patient being classified by the computing device 102. In some aspects, the user 142 may also be a patient at the healthcare facility 150.

Furthermore, it should be understood that the medical data may be received by the computing device 102 from more than one of the data sources 170, the user computing device 140, and/or the database 118. Moreover, it should be understood that although the example of FIG. 1 illustrates only one of each of the user computing device 140 and user 142, any number of user computing devices 140 and/or users 140 may be used.

The medical data (e.g., held by any of the data sources 170, and/or the database 118) may include any medical data. Examples of the medical data include: disease classification codes (e.g., International Classification of Diseases (ICD) codes); Health and Human Services (HHS)-Hierarchical Condition Categories (HHS-HCC) data; Centers for Medicare & Medicaid Services (CMS)-HCC data; information of a hospice stay; dates of death; information of stays at a skilled nursing facility (SNF); information of stays at a residential boarding house (BH); Chronic Conditions Wearhouse (CCW) data; ages of the patients; numbers of emergency department (ED) visits; indications of lines of business (LOBs) being any of commercial, Medicare, or Medicare; information of a diagnosis related group (DRG); and/or information of a behavioral health inpatient stay. In addition, the medical data may be respective medical data; that is, the medical data corresponds to individual patients, and thus may be used to classify the individual patients.

### Example Categories

The systems and methods described herein may divide patients (e.g., medical patients, etc.) into categories. In some embodiments, the categories are hierarchical categories. However, in other embodiments, the categories are not hierarchical. In addition, although some embodiments use a specific number of categories, any number of categories and/or subcategories may be used.

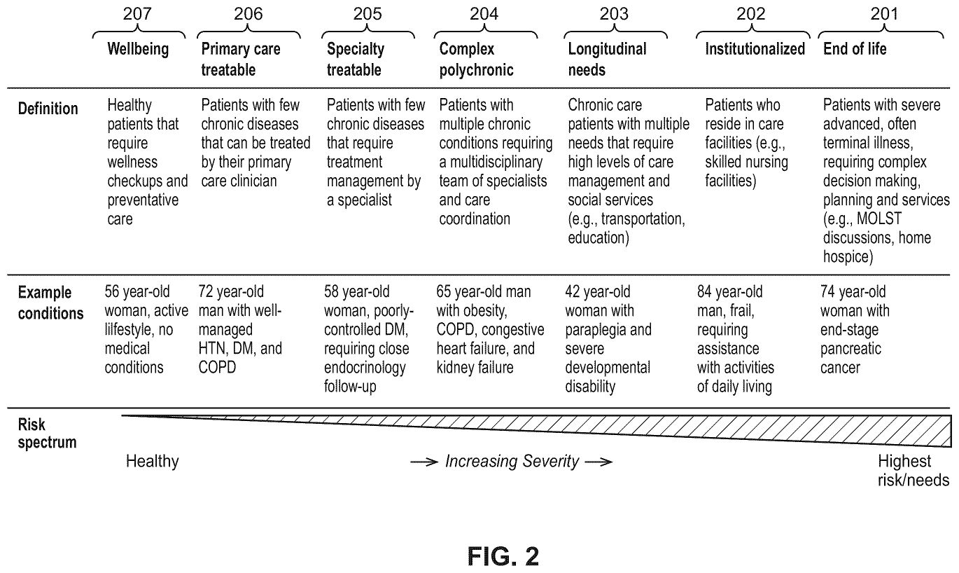

FIG. 2 illustrates an example of seven hierarchical categories. As will be seen, the illustrated example includes a range from a most severe category 201 (e.g., an end of life category (EOL)) to a least severe category 207 (e.g., a wellbeing category); and thus, the example categories illustrated in the example of FIG. 2 are hierarchical.

More specifically, in the example of FIG. 2, the EOL category 201 includes patients with severe advanced, often terminal illness, requiring complex decision making, planning and services (e.g., medical orders for life sustaining treatment (MOLST) discussions, home hospice, etc.). In one example, the EOL category includes a 74 year-old woman with end-stage pancreatic cancer.

The institutionalized category 202 (e.g., a second most severe category in the illustrated example) may include, for example, patients who reside in care facilities (e.g., skilled nursing facilities). In one example, the institutionalized category 202 includes a 84 year-old man who is frail and requires assistance with activities of daily living.

The longitudinal needs category 203 (e.g., a third most severe category in the illustrated example) may include, for example, chronic care patients with multiple needs that require high levels of care management and social services (e.g., transportation, education, etc.). In one example, the longitudinal needs category 203 includes a 42 year-old woman with paraplegia and severe developmental disability.

The complex polychronic category 204 (e.g., a fourth most severe category in the illustrated example) may include, for example, patients with multiple chronic conditions requiring a multidisciplinary team of specialists and care coordination. In one example, the complex polychronic category 204 includes a 65 year-old man with obesity, chronic obstructive pulmonary disease (COPD), congestive heart failure, and kidney failure.

The specialty treatable category 205 (e.g., a fifth most severe category in the illustrated example) may include, for example, patients with few chronic diseases that require treatment management by a specialist. In one example, the specialty treatable category 205 includes a 58 year-old woman with poorly-controlled diabetes mellitus (DM), and requiring close endocrinology follow-up.

The primary care treatable category 206 (e.g., a sixth most severe category in the illustrated example) may include, for example, patients with few chronic diseases that require treatment management by a specialist. In one example, the primary care treatable category 206 includes a 72 year-old man with well-managed hypertension (HTN), DM, and COPD.

The wellbeing category 207 (e.g., a least severe category in the illustrated example) may include, for example, healthy patients that require wellness checkups and preventative care. In one example, the wellbeing category 207 includes a 56 year-old woman, active lifestyle, no medical conditions.

In operation, the patient classifier 124 receives the respective medical data, and classifies patients into the categories based on the respective medical data. For example, the patient classifier 124 may classify the patients into hierarchical categories ranging from most severe to least severe, such as EOL category 201, institutionalized category 202, longitudinal needs category 203, complex polychronic category 204, specialty treatable category 205, primary care treatable category 206, and wellbeing category 207.

The organizational predictor 126, in operation, predicts an organizational improvement, such as an optimal overall staffing level of a healthcare facility, an optimal staffing level for a particular type of caregiver, an optimal ratio of a first type of caregiver to a second type of caregiver, or an amount of financing that will be required for a particular category. In particular, the organizational predictor 126 uses the classifications made by the patient classifier 124 to predict the organizational improvement. For example, the organizational predictor 126, may use numbers of patients within a predetermined number of most severe categories to predict an optimal number (or range of numbers) of MAs at the healthcare facility 150 for a given time period (e.g., day, range of days, week, etc.).

The clinical operational predictor 128, in operation, predicts a clinical operational improvement, such as an optimal bundle of services for a patient, or an improvement in a care pathway. More specifically, the clinical operational predictor 128 uses the classifications made by the patient classifier 124 to predict the clinical operational improvement. For example, the clinical operational predictor 128 may predict, based on a patient's classification, that there will be an improvement in outcome if a step in a care pathway is modified. For instance, if the patient is in a more severe category, the clinical operational predictor 128 may predict that the patient should spend more time with an APP. On the other hand, if the patient is in a less severe category, the clinical operational predictor 128 may predict that the patient should spend less time with the APP, and/or more time with a PCP.

The machine learning training application 130, in operation, may train machine learning algorithm(s) that are used by other components of the computing device 102. In one example, machine learning training application 130 trains a machine learning algorithm to classify subjects into categories (e.g., a machine learning algorithm used by the patient classifier 124). In another example, the instructions may train a machine learning algorithm to predict organizational improvements (e.g., a machine learning algorithm used by the organizational predictor 126). In yet another example, the instructions may train a machine learning algorithm to predict clinical operational improvements (e.g., a machine learning algorithm used by the clinical operational predictor 128).

### Example Organizational Predictions

As will be seen, organizational predictions made by the techniques described herein may bring many benefits to healthcare providers.

In one example, the organizational predication may be a prediction of an optimal staffing level of a particular type of caregiver (e.g., a PCP, an advanced practice provider (APP), a registered nurse (RN), a medical assistant (MA), a care manager (CM), etc.) at a healthcare facility (e.g., a hospital, etc.).

For instance, the organizational predictor 126 may analyze the numbers of patients in categories classified by the patient classifier 124, and determine that there are a large number of patients in less severe categories (e.g., the wellbeing category 207). In response to this determination, the organizational predictor 126 may determine a large number of PCPs to be optimal.

On the other hand, if there are a large number of patients in more severe categories (e.g., the institutionalized category 202, and/or the EOL category 201), organizational predictor 126 may determine a large number of APPs to be optimal. Advantageously, this allows for much more accurate predictions of optimal caregiver levels than is possible based on other techniques. For example, a technique that predicts optimal PCP or APP staff levels based only on overall incoming patient numbers is not as accurate. In particular, such a technique based only overall incoming patient numbers would not account for imbalances in the severity of the conditions of the incoming patients (e.g., when there are more patients with more severe health conditions, more APPs are needed; whereas, when there are more patients with less severe health conditions, more PCPs are needed).

To this end, the organizational predictor 126 may also make predictions of optimal ratios of types of caregivers. For instance, if there are a large number of patients in less severe categories (e.g., the wellbeing category 207), the organizational predictor 126 may determine a high ratio of PCPs to APPs to be optimal. In contrast, if there are a large number of patients in more severe categories (e.g., the institutionalized category 202, and/or the EOL category 201), the organizational predictor 126 may determine a low ratio of PCPs to APPs to be optimal.

In another example, the organizational prediction may be a prediction of an optimal overall staffing level of a healthcare facility. For instance, the organizational predictor 126 may analyze the numbers of patients in categories classified by the patient classifier 124, and determine that there are larger numbers of patients in more severe categories than in less severe categories. In response, the organizational predictor 126 may determine that a higher overall staffing level is optimal. On the other hand, if there are lower numbers of patients in more severe categories, the organizational predictor 126 may determine that a lower overall staffing level is optimal.

Moreover, the organizational predictor 126 may predict any of the optimal overall staffing level, optimal staffing level for a particular type of caregiver, and/or optimal ratio of types of caregivers along with a corresponding time period (e.g., a day, a week, a month, etc.). For instance, the organizational predictor 126 may use known schedules of patients at a healthcare facility 150 to determine the time periods. In one illustrative example, the organizational predictor 126 may determine that, during a particular week, there are large numbers of patients in more severe categories scheduled to be at the healthcare facility 150. In response to this determination, the organizational predictor 126 may predict a high optimal overall staffing level.

In yet another example, the organizational predictor 126 may predict amounts of financing that will be required for the categories. For example, the organizational predictor 126 may determine a number of patients in each category. The organizational predictor 126 may then use a lookup table to determine a predicted amount of financing that will be required for each category.

Additionally or alternatively, the organizational predictor 126 may use a machine learning algorithm to determine the predicted amount of required financing. For example, the machine learning training application 130 may train a machine learning algorithm to be used by the organizational predictor 126 for this purpose. Specifically, the machine learning training application 130 may receive historical data of: patients, categories that the patients are in, costs of the patients, etc. The machine learning training application 130 may then train a machine learning algorithm using this historical data (e.g., train using a supervised, unsupervised, or semi-supervised learning process) to predict financing for each category. The organizational predictor 126 may then use this trained machine learning algorithm to predict the required amounts of financing for each category.

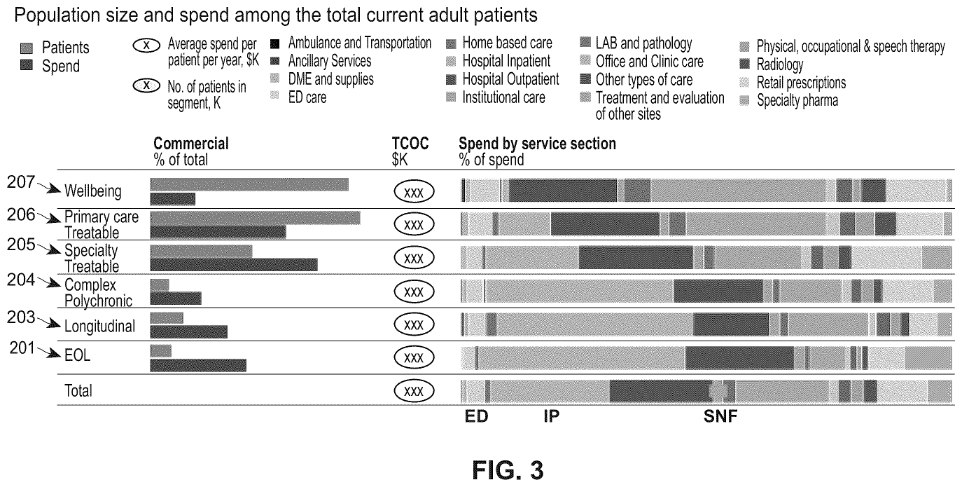

In this regard, FIG. 3 shows an example of spending based on category. In the illustrated example, the EOL category 201 (e.g., a most severe category) has a disproportionately high (based on the percentages of patients in each category) percentage of predicted spend compared to the less severe categories.

As further illustrated in the example of FIG. 3, spending for service sections may be calculated and displayed. Examples of service sections include: ambulance and transportation; ancillary services; durable medical equipment (DME) and supplies; emergency department (ED) care; home based care; hospital inpatient care; hospital outpatient care; institutional care; lab and/or pathology; office and/or clinic care; treatment and evaluation of other sites; physical, occupational, and/or speech therapy; radiology; retail prescriptions; and specialty pharma. Thus, as illustrated in the example of FIG. 3, calculated spending may be shown based on category (e.g., categories 201-207), and/or service section.

In some embodiments, the organizational predictor 126 calculates the spending (e.g., for the categories and/or service sections) prior to or as part of predicting future spending (e.g., again for the categories and/or service sections). To predict spending for a particular service section, the organizational predictor 126 may start by determining a number of patients in each category. The organizational predictor 126 may then use a lookup table to determine a predicted amount of financing for each service section.

Additionally or alternatively, the organizational predictor 126 may use a machine learning algorithm to determine the predicted amount of required financing for each service section by patient category. For example, the machine learning training application 130 may train a machine learning algorithm to be used by the organizational predictor 126. In particular, the machine learning training application 130 may receive historical data of: patients, categories that the patients are in, costs of the patients per service section, etc. The machine learning training application 130 may then train a machine learning algorithm using this historical data (e.g., train using a supervised, unsupervised, or semi-supervised learning process) to predict financing for each service section. The organizational predictor 126 may then use this trained machine learning algorithm to predict the required amounts of financing for each service section.

### Example Clinical Operational Predictions

The classifications of the patients made by the patient classifier 124 may also be used by the clinical operational predictor 128 to make clinical operational predictions. For instance, the clinical operational predictor 128 may predict an optimal bundle of services (e.g., including types of touchpoints, time durations for the types of touchpoints, and numbers of times per bundle for the types of touchpoints) for a patient corresponding to a category classification of the patient.

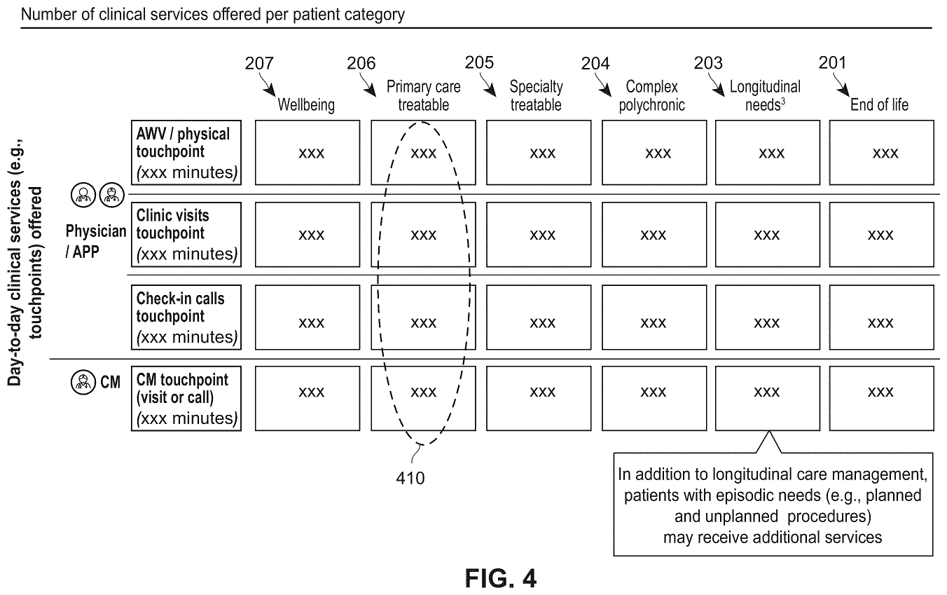

FIG. 4 illustrates example bundles for example categories. With reference thereto, each example category (e.g., EOL category 201, longitudinal needs category 203, complex polychronic category 204, specialty treatable category 205, primary care treatable category 205, primary care treatable category 206, wellbeing category 207) has an associated bundle of services. For example, bundle 410 is associated with the primary care treatable category 206, and includes: a predetermined number of annual wellness visits (AWVs) or physical touchpoints (with a time duration of a predetermined number of minutes); a predetermined number of clinic visit touchpoints (with a time duration of a predetermined number of minutes); a predetermined number of check in call touchpoints (with a time duration of a predetermined number of minutes); and a predetermined number of CM touchpoints (each with a time duration of a predetermined number of minutes).

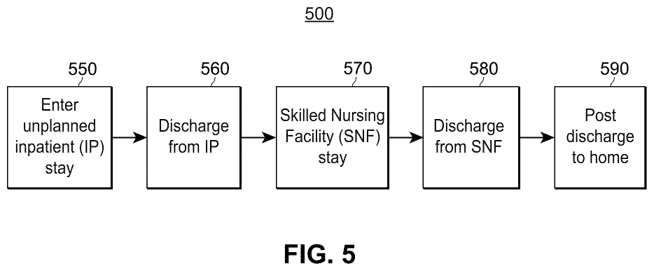

In another example, the clinical operational predictor 128 may predict that modifying a care pathway for a patient based on the patient's category classification improves patient outcome, expenditures for the patient, and/or expenditures for the medical facility. In this regard, FIG. 5 shows an example care pathway 500 of a patient in an unplanned inpatient (IP) admission. Broadly speaking, a care pathway may include a schedule of visits with patients. In some examples, the care pathway includes phases; and in some further examples, the phases include steps.

For instance, the example pathway 500 includes phases 550, 560, 570, 580, 590. More specifically, in the illustrated example, at phase 550, the patient enters an unplanned IP stay. At phase 560, the patient is discharged from the IP stay. At phase, 570, the patient enters a stay at a SNF. At phase 580, the patient is discharged from the SNF. In phase 590, the patient is at her home in a post discharge phase. Here, any of the phases may include steps within the phase, such as visits with a PCP, a CM, a nurse, a specialist, a pharmacist, a social worker, etc. In some examples, the steps include associated time periods. For instance, a step within the post discharge phase 590 may be a 30-day CM post-admission program.

However, in accordance with the techniques described herein, the clinical operational predictor 128 may predict that modifying the care pathway 500 improves patient outcome, expenditures for the patient, and/or expenditures for the medical facility. For example, the time periods associated with steps may be modified. For instance, if there is a 30-day CM post-admission program in a post discharge phase, the 30-day period may be increased or decreased. For example, patients in a more severe category may have this duration increased (e.g., a patient in the longitudinal needs category 203 may have this duration increased to 60 days). On the other hand, a patient in a less severe category may have this duration decreased (e.g., a patient in the primary care treatable category 206 may have this duration decreased to a week).

In another example, the modification (e.g., predicted by the clinical operational predictor 128) may comprise removing step(s) and/or phase(s) of the care pathway. For example, a patient in a less severe category may have a step and/or phase of the care pathway removed (e.g., a patient in the wellbeing category 207 may have step of a CM post-admission removed entirely because, for example, it is predicted that patients in this category will not significantly benefit from a CM post-admission program).

In yet another example, a step and/or phase may be added to a care pathway. For example, an intervention may be added to a care pathway. For instance, if both a caregiver suspects a patient has a particular disease and the patient is in a more severe category, an intervention (e.g., taken in an attempt to prevent or treat the suspected disease) may be placed in a care pathway.

In yet another example, the modification to the care pathway may be based on other data in addition to the patient category classification. For instance, the modification may be based on a consumer type that the patient is considered to be. For example, the patient may be a consumer type of “tech adopter,” and have a patient category of complex polychronic. Based on this consumer type and patient category, the modification may be an addition of a step to send periodic email or text message reminders during a specified time period (e.g., a post discharge time period) to check insulin or blood pressure. In another example, if the patient has a consumer type of “prefers mailers” and a patient category of EOL, the patient may be mailed reminders to visit her PCP or other healthcare provider.

### Example Methods

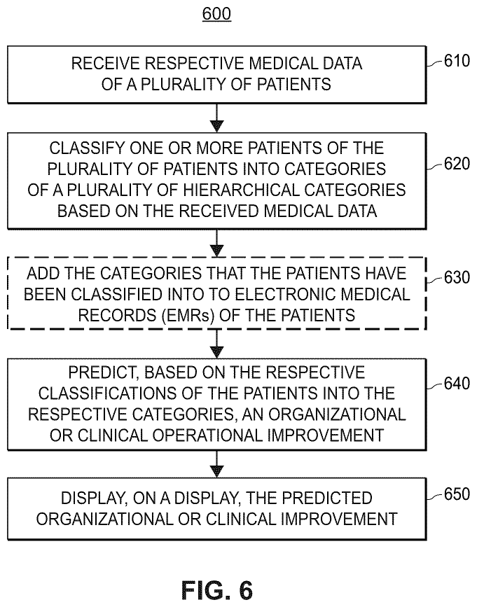

FIG. 6 shows an example method 600 of classifying medical patients, according to some aspects. For illustrative purposes, the following discussion will refer to the one or more processors 120 of the computing device 102 as performing the blocks of the method 600. However, it should be understood that any other component (e.g., one or more processors of any of the data sources 170, etc.) may perform any of the blocks instead of and/or in conjunction with the one or more processors 120.

The method 600 may include receiving, with the patient classifier 124, respective medical data of a plurality of subjects (block 610). As discussed above, the medical data may be received from any suitable source, such as a data source 170, the user computing device 140, and/or the database 118. As also discussed above, examples of the medical data include a disease classification code (e.g., an ICD code); HHS-HCC data; CMS-HCC data; information of a hospice stay; dates of death; information of stays at a SNF; information of stays at a residential BH; CCW data; ages of the patients; numbers of ED visits; indications of LOBs being any of commercial, Medicare, or Medicare; information of a DRG; and/or information of a behavioral health inpatient stay.

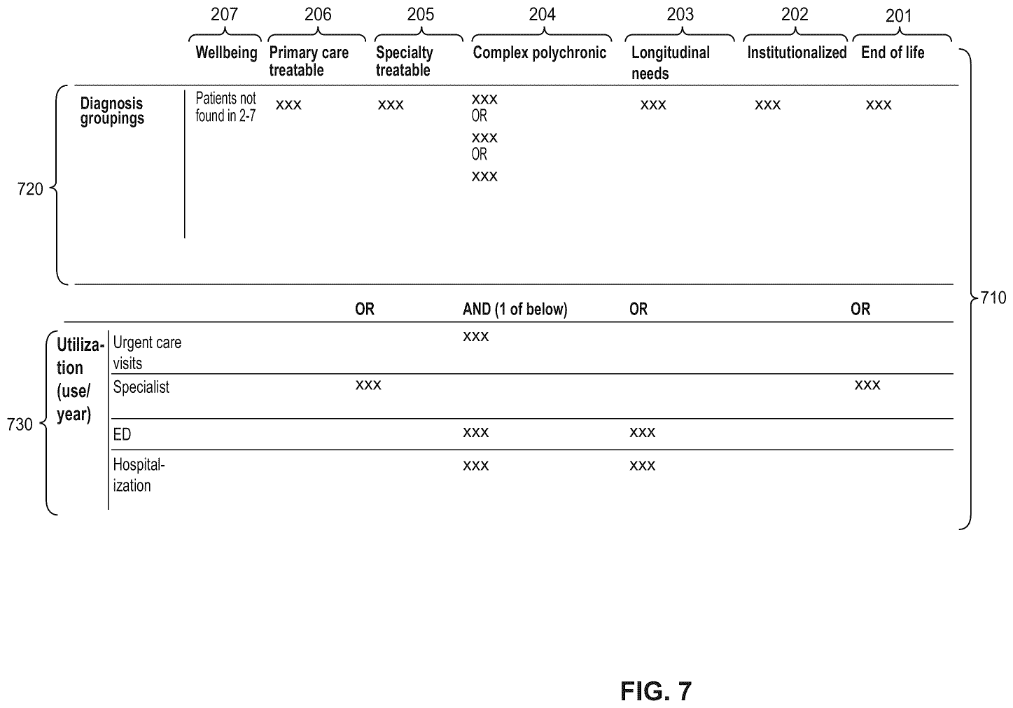

The method 600 may include classifying patients of the plurality of patients into respective categories of a plurality of hierarchical categories based on the received medical data (block 620). In some examples, the patients are classified by the patient classifier 124 in a hierarchical fashion based on a set of rules. In this regard, by way of illustrative example, FIG. 7 shows example categories 201-207 and example criteria 710. In some embodiments, the patient classifier 124 may first evaluate a patient with respect to the criteria for a first category (which, in some embodiments, may be a most severe category, e.g., EOL category 201 in the example of FIG. 7).

If the criteria for the first category is met, the patient may be classified into the first category. If not, the patient classifier 124 may the evaluate a patient with respect to the criteria for the second category (which, in some embodiments, may be a second most severe category, e.g., institutionalized category 202 in the example of FIG. 7). If the criteria for the second most severe category is met, the patient may be classified into the second most severe category. If not, the patient classifier 124 may evaluate the patient with respect to the criteria for the third category, and so on until the patient is classified into a category. In some embodiments, the criteria for the final category is simply that the patient has not been classified into any of the other categories.

To illustrate, in one exemplary implementation of the example of FIG. 7, first, the patient classifier 124 evaluates the criteria for the EOL category 201. This example criteria for the EOL category 201 is that: the patient has metastatic cancer, the patient has cancer with a low five year survival rate, the patient has end stage liver disease and sequalae, the patient has rejected a major organ transplant, the patient has died within a claims year, or the patient has received hospice treatment (e.g., the received medical data includes hospice taxonomy codes). (Regarding the criteria that the patient has died within a claims year, it should be understood that, in some embodiments, a category (or categories) may include deceased patients.) If the criteria for the EOL category 201 is met, the patient is classified into the EOL category. If not, the patient classifier 124 then evaluates the patient with respect to the criteria for the institutionalized category 202 (e.g., the second most severe category).

In the illustrated example, the criteria for the institutionalized category 202 is that the patient was in a residential behavioral health or SNF for a prolonged period (e.g., a week, a month, etc.). If this criteria is met, the patient classifier 124 classifies the patient into the institutionalized category 202. If not, the patient classifier 124 evaluates the patient with respect to the criteria for the longitudinal needs category 203. In the illustrated example, the criteria for the longitudinal needs category 203 is that the patient has: severe musculoskeletal impairments, an intellectual disability, an addiction disorder, a potentially debilitating neurologic/psychiatric disease, a super-utilization of the ED (e.g., 3 ED visits within the past year), or an increased behavioral health hospitalization utilization (e.g., a trend of increasing visits to a behavioral health specialist). The one or more processors 120 then continue to evaluate the patient with respect to the criteria for each of the remaining categories until the patient is classified. It should be understood that, in the illustrated example, the patient will be classified in any event because the criteria for the wellbeing category 207 (e.g., the final category) is simply that the patient was not classified into any of the other categories.

It should be understood that FIG. 7 is only an example, and other implementations are contemplated. For instance, in other examples, the patient classifier 124 evaluates the patient with respect to criteria for a first category, where the first category is a least severe category. In other examples, the evaluations are done in an order without respect to a hierarchy of severity.

Moreover, as further illustrated in the example of FIG. 7, the criteria may include diagnosis grouping criteria 720 and/or utilization criteria 730. As further illustrated in the example of FIG. 7, to form the criteria, the diagnosis grouping criteria 720 and utilization criteria 730 may be concatenated together by an “and” or an “or.”

In addition, with respect to the example of FIG. 7, it should be understood that there are two separate aspects to “hierarchy.” First, there is that the categories themselves are hierarchical (e.g., the example categories 201-207 range from most severe to least severe). Second, there is that the classification process is hierarchical (e.g., criteria for the most severe category is evaluated before proceeding to evaluate the criteria for the second most severe category, and so on). And here, when the classification process is hierarchical, the system advantageously achieves technical advantages. In particular, by hierarchically classifying the patients, the system avoids having to evaluate the criteria for every patient (e.g., if a patient is classified into the EOL category 201, the patient does not need to be evaluated with respect to the criteria for the other categories). Advantageously, this saves computational resources, and improves system run time. This advantage becomes more significant when large numbers of patients are being classified and/or when the classification criteria requires large amounts of data. For example the technical advantage becomes even more pronounced if the criteria involves all of IDC codes, HHS-HCC, and CMS-HCC data rather than only IDC codes.

Additionally or alternatively, in some embodiments, the patient classifier 124 may classify the patients using a machine learning algorithm. For example, a machine learning algorithm may be trained (e.g., by the machine learning training application 130) based on historical medical data (e.g., historical medical data including any of a disease classification code [e.g., an ICD code]; HHS-HCC data; CMS-HCC data; information of a hospice stay; dates of death; information of stays at a SNF; information of stays at a residential BH; CCW data; ages of the patients; numbers of ED visits; indications of LOBs being any of commercial, Medicare, or Medicare; information of a DRG; and/or information of a behavioral health inpatient stay), and known classifications of patients corresponding to the medical data. Subsequently, the medical data received by the patient classifier 124 at block 610 may be input into the trained machine learning algorithm at block 620 to classify patients. Any type of suitable machine learning algorithm (e.g., convolutional neural network, random forest, etc.) may be used.

Further at block 620, the patient classifier 124 may classify the patients into subcategories as well. For example, within an EOL category, there may be a deceased subcategory (e.g., as determined by receiving medical data including a date of death of a patient). In another example, a category may include a subcategory that indicates a prediction that the patient will likely soon transition to a more severe category. For instance, if a category has a criteria that a patient had three to five ED visits within the past month, and the patient had five ED visits, the patient may be placed in the category as well as placed in a subcategory that indicates that the patient will likely transition to a more severe category.

In another example, the patient classifier 124 may classify the patient into a subcategory indicating that the patient recently transitioned to this category from a more severe category (thus potentially indicating health improvement of the patient). For example, if the patient had, within a prespecified time period, previously been classified in a more severe category, the patient may be placed in a subcategory indicating the recent transition.

In some embodiments, the subcategories (or some of the subcategories) may be hierarchical. In other embodiments, the subcategories are not hierarchical.

In some embodiments, there are categories and subcategories that distinguish between adult and pediatric patients. For example, there may be a first category for adult patients, and a second category for pediatric patients. In some implementations of this example, each of the first and second categories may have subcategories corresponding to the example categories 201-207 (e.g., each of the first and second categories have sub categories of: EOL, institutionalized, longitudinal needs, complex polychronic, specialty treatable, primary care treatable, and/or wellbeing). In some implementations of this, though, the subcategories are not the same for the first and second categories. For example, it is less common for pediatric patients to be institutionalized so an institutionalized subcategory is not included for the pediatric category.

Additionally or alternatively, in some embodiments, the classification may only be applied to certain groups of patients. For example, the classification may only be applied to a group of people in a certain age range. In this way, in some embodiments, the entire system may be for adult patients, pediatric patients, etc. For instance, the classification may only be done on patients with an age of less than 18 so that the system is a pediatric system. To further illustrate, some example implementations of a pediatric system: (i) include that the classification is only done on patients with an age of less than 18, and (ii) do not include an institutionalized category. In some examples, patients with an age of 18 or older are classified within adult hierarchical categories; and patients with an age of less than 18 are classified within pediatric hierarchical categories.

The method 600 may optionally include the patient classifier 124 adding the classifications to electronic medical records (EMRs) of the patients (block 630). Advantageously, in some implementations, this allows for technical improvements. For example, a user may wish to predict an optimal staffing level of a particular type of caregiver. Here, if the categories have been added to the EMRs of the patients, the organizational predictor 126 is able to simply draw the data from the EMRs patients of the healthcare provider 150 for which the prediction is being made rather than having the patient classifier 124 classify every patient based on medical data.

The method may include the organizational predictor 126 predicting an organizational improvement based on the respective classifications, or the clinical operational predictor 128 predicting a clinical operational improvement based on the respective classifications (block 640).

Examples of organizational improvements are discussed above, and include: an optimal overall staffing level of a healthcare facility; an optimal staffing level for a particular type of caregiver; an optimal ratio of a first type of caregiver to a second type of caregiver; and an amount of financing that will be required for a hierarchical category of the plurality of hierarchical categories.

Additionally or alternatively to predicting a clinical or organizational improvement, the one or more processors (e.g., with the clinical operational predictor 128 or any other component) may calculate spending for categories and/or service sections (e.g., as in the related example of FIG. 3).

Examples of clinical operational improvements are also discussed above, and include: a decision to implement a bundle of services for a patient, a decision to modify a care pathway for a patient, and a decision to place an intervention in a care pathway of a patient.

The method may include the computing device 102 displaying the predicted organizational or clinical operational improvement (block 650). For example, the predicted organizational or clinical operational improvement may be displayed on display 139. Additionally or alternatively, the computing device 102 may display the calculated spending (e.g., as in the related example of FIG. 3).

Further regarding the example flowcharts provided above, it should be noted that all blocks are not necessarily required to be performed. Moreover, additional blocks may be performed although they are not specifically illustrated in the example flowcharts.

### Additional Exemplary Embodiments

Aspect 1. A computer-implemented method for classifying medical patients, the method comprising:
receiving, via one or more processors, respective medical data of a plurality of patients;
classifying, via the one or more processors, one or more patients of the plurality of patients into respective categories of a plurality of hierarchical categories based on the received respective medical data;
predicting, via the one or more processors, based on the respective classifications of the patients into the respective categories, an organizational or clinical operational improvement; and
displaying, via the one or more processors, the predicted organizational or clinical operational improvement.

Aspect 1A. A computer-implemented method for classifying medical patients, the method comprising:
receiving, via one or more processors, respective medical data of a plurality of patients;
classifying, via the one or more processors, one or more patients of the plurality of patients into respective categories of a plurality of hierarchical categories based on the received respective medical data;
calculating, via the one or more processors, spending for each of the respective categories; and
displaying, via the one or more processors, the calculated spending for each of the respective categories.

Aspect 2. The computer-implemented method of aspect 1, wherein the classifying comprises:
determining, via the one or more processors, if a patient of the plurality of patients meets criteria of a most severe health category of the plurality of hierarchical categories; and
if the patient does not meet the criteria of the most severe health category, determining, via the one or more processors, if the patient should be placed in a second most severe health category.

Aspect 3. The computer-implemented method of any one of aspects 1-2, wherein the received respective medical data comprises at least one of:
International Classification of Diseases (ICD) codes;
Health and Human Services (HHS)-Hierarchical Condition Categories (HHS-HCC) data;
Centers for Medicare & Medicaid Services (CMS)-HCC data; information of a hospice stay;
dates of death;
information of stays at a skilled nursing facility (SNF);
information of stays at a residential boarding house (BH);
Chronic Conditions Wearhouse (CCW) data;
ages of the patients;
numbers of emergency department (ED) visits;
indications of lines of business (LOBs) being any of commercial, Medicare, or Medicare;
information of a diagnosis related group (DRG); or
information of a behavioral health inpatient stay.

Aspect 4. The computer-implemented method of any one of aspects 1-3, wherein the received respective medical data comprises at least one of:
a disease classification code, or information of a stay in a healthcare facility.

Aspect 5. The computer-implemented method of any one of aspects 1-4, wherein the classifying comprises:
classifying, via the one or more processors, with a trained machine learning algorithm, the patients of the plurality of patients into the respective categories of the plurality of hierarchical categories.

Aspect 6. The computer-implemented method of any one of aspects 1-5, further comprising:
adding, via the one or more processors, the categories that the patients have been classified into to electronic medical records (EMRs) of the patients.

Aspect 7. The computer-implemented method of any one of aspects 1-6, wherein the plurality of patients are pediatric patients, and the plurality of hierarchical categories are a plurality of pediatric hierarchical categories.

Aspect 8. The computer-implemented method of any one of aspects 1-7, wherein at least one category of the plurality of hierarchical categories includes a plurality of hierarchical subcategories.

Aspect 9. The computer-implemented method of any one of aspects 1-8, wherein predicting the organizational improvement comprises predicting at least one of:
an optimal overall staffing level of a healthcare facility;
an optimal staffing level for a particular type of caregiver;
an optimal ratio of a first type of caregiver to a second type of caregiver; or
an amount of financing that will be required for a hierarchical category of the plurality of hierarchical categories.

Aspect 10. The computer-implemented method of any one of aspects 1-9, wherein predicting the organizational improvement comprises predicting an optimal staffing level of a particular type of caregiver, the particular type of caregiver being one of:
a primary care physician (PCP);
an advanced practice provider (APP);
a registered nurse (RN);
a medical assistant (MA); or
a care manager (CM).

Aspect 11. The computer-implemented method of any one of aspects 1-10, wherein predicting the organizational improvement comprises predicting an amount of financing that will be required for a service section, and the service section comprises at least one of:
ambulance and transportation;
ancillary services;
durable medical equipment (DME) and supplies;
emergency department (ED) care;
home based care;
hospital inpatient care;
hospital outpatient care;
institutional care;
lab and/or pathology;
office and/or clinic care;
treatment and evaluation of other sites;
physical, occupational, and/or speech therapy;
radiology;
retail prescriptions; or
specialty pharma.

Aspect 12. The computer-implemented method of any one of aspects 1-11, wherein predicting the clinical operational improvement comprises predicting an optimal bundle of services for a patient of the plurality of patients by assigning the patient a bundle of services corresponding to a category classification of the patient.

Aspect 13. The computer-implemented method of any one of aspects 1-12, wherein predicting the clinical operational improvement comprises predicting bundles of services for patients of the plurality of patients corresponding to category classifications of the patients, and wherein the bundles comprise: (i) types of touchpoints, (ii) time durations for the types of touchpoints, and (iii) numbers of times per bundle for the types of touchpoints.

Aspect 14. The computer-implemented method of any one of aspects 1-13, wherein predicting the clinical operational improvement comprises modifying a care pathway and/or placing an intervention in the care pathway, the care pathway comprising a schedule of visits with healthcare providers.

Aspect 15. A computer system for classifying medical patients, the computer system comprising one or more processors configured to:
receive respective medical data of a plurality of patients;
classify one or more patients of the plurality of patients into respective categories of a plurality of hierarchical categories based on the received respective medical data;
predict, based on the respective classifications of the patients into the respective categories, an organizational or clinical operational improvement; and
display, on a display, the predicted organizational or clinical operational improvement.

Aspect 16. The computer system of aspect 15, wherein the one or more processors are further configured to classify the patients by:
determining if a patient of the plurality of patients meets criteria of a first category of the plurality of hierarchical categories; and
if the patient does not meet the criteria of the first category, determining if the patient should be placed in a second category.

Aspect 17. The computer system of any one of aspects 15-16, wherein the received respective medical data comprises at least one of:
a disease classification code, or information of a stay in a healthcare facility.

Aspect 18. A computing device for classifying medical patients, the computing device comprising:
one or more processors; and
one or more memories;
   the one or more memories having stored thereon computer-executable instructions that, when executed by the one or more processors, cause the computing device to:
receive respective medical data of a plurality of patients;
classify one or more patients of the plurality of patients into respective categories of a plurality of hierarchical categories based on the received respective medical data;
predict, based on the respective classifications of the patients into the respective categories, an organizational or clinical operational improvement; and
display, on a display, the predicted organizational or clinical operational improvement.

Aspect 19. The computing device of aspect 18, the one or more memories having stored thereon computer executable instructions that, when executed by the one or more processors, cause the computing device to classify the patients by:
determining if a patient of the plurality of patients meets criteria of a most severe health category of the plurality of hierarchical categories; and
if the patient does not meet the criteria of the most severe health category, determining if the patient should be placed in a second most severe health category.

Aspect 20. The computing device of any one of aspects 18-19, the one or more memories having stored thereon computer executable instructions that, when executed by the one or more processors, cause the computing device to predict the organizational improvement by predicting an optimal staffing level for a particular type of caregiver.

### Other Matters

Additionally, certain embodiments are described herein as including logic or a number of routines, subroutines, applications, or instructions. These may constitute either software (code embodied on a non-transitory, tangible machine-readable medium) or hardware. In hardware, the routines, etc., are tangible units capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

In various embodiments, a hardware module may be implemented mechanically or electronically. For example, a hardware module may comprise dedicated circuitry or logic that is permanently configured (e.g., as a special-purpose processor, such as a field programmable gate array (FPGA) or an application-specific integrated circuit (ASIC) to perform certain operations. A hardware module may also comprise programmable logic or circuitry (e.g., as encompassed within a general-purpose processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

Accordingly, the term “hardware module” should be understood to encompass a tangible entity, be that an entity that is physically constructed, permanently configured (e.g., hardwired), or temporarily configured (e.g., programmed) to operate in a certain manner or to perform certain operations described herein. Considering embodiments in which hardware modules are temporarily configured (e.g., programmed), each of the hardware modules need not be configured or instantiated at any one instance in time. For example, where the hardware modules comprise a general-purpose processor configured using software, the general-purpose processor may be configured as respective different hardware modules at different times. Software may accordingly configure a processor, for example, to constitute a particular hardware module at one instance of time and to constitute a different hardware module at a different instance of time.

Hardware modules can provide information to, and receive information from, other hardware modules. Accordingly, the described hardware modules may be regarded as being communicatively coupled. Where multiple of such hardware modules exist contemporaneously, communications may be achieved through signal transmission (e.g., over appropriate circuits and buses) that connect the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it is communicatively coupled. A further hardware module may then, at a later time, access the memory device to retrieve and process the stored output. Hardware modules may also initiate communications with input or output devices, and can operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods or routines described herein may be at least partially processor-implemented. For example, at least some of the operations of a method may be performed by one or more processors or processor-implemented hardware modules. The performance of certain of the operations may be distributed among the one or more processors, not only residing within a single machine, but deployed across a number of machines. In some example embodiments, the processor or processors may be located in a single location (e.g., within a home environment, an office environment or as a server farm), while in other embodiments the processors may be distributed across a number of geographic locations.

Furthermore, the patent claims at the end of this patent application are not intended to be construed under 35 U.S.C. § 112(f) unless traditional means-plus-function language is expressly recited, such as “means for” or “step for” language being explicitly recited in the claim(s). The systems and methods described herein are directed to an improvement to computer functionality, and improve the functioning of conventional computers.

What is claimed:

1. A computer-implemented method for classifying medical patients, the method comprising:
receiving, via one or more processors, respective medical data of a plurality of patients;
classifying, via the one or more processors, one or more patients of the plurality of patients into respective categories of a plurality of hierarchical categories based on the received respective medical data;
predicting, via the one or more processors, based on the respective classifications of the patients into the respective categories, an organizational or clinical operational improvement; and
displaying, via the one or more processors, the predicted organizational or clinical operational improvement.

2. The computer-implemented method of claim 1, wherein the classifying comprises:
determining, via the one or more processors, if a patient of the plurality of patients meets criteria of a most severe health category of the plurality of hierarchical categories; and
if the patient does not meet the criteria of the most severe health category, determining, via the one or more processors, if the patient should be placed in a second most severe health category.

3. The computer-implemented method of claim 1, wherein the received respective medical data comprises at least one of:
International Classification of Diseases (ICD) codes;
Health and Human Services (HHS)-Hierarchical Condition Categories (HHS-HCC) data;
Centers for Medicare & Medicaid Services (CMS)-HCC data;
information of a hospice stay;
dates of death;
information of stays at a skilled nursing facility (SNF);
information of stays at a residential boarding house (BH);
Chronic Conditions Wearhouse (CCW) data;
ages of the patients;
numbers of emergency department (ED) visits;
indications of lines of business (LOBs) being any of commercial, Medicare, or Medicare;
information of a diagnosis related group (DRG); or
information of a behavioral health inpatient stay.

4. The computer-implemented method of claim 1, wherein the received respective medical data comprises at least one of:
a disease classification code, or information of a stay in a healthcare facility.

5. The computer-implemented method of claim 1, wherein the classifying comprises:
classifying, via the one or more processors, with a trained machine learning algorithm, the patients of the plurality of patients into the respective categories of the plurality of hierarchical categories.

6. The computer-implemented method of claim 1, further comprising:
adding, via the one or more processors, the categories that the patients have been classified into to electronic medical records (EMRs) of the patients.

7. The computer-implemented method of claim 1, wherein the plurality of patients are pediatric patients, and the plurality of hierarchical categories are a plurality of pediatric hierarchical categories.

8. The computer-implemented method of claim 1, wherein at least one category of the plurality of hierarchical categories includes a plurality of hierarchical subcategories.

9. The computer-implemented method of claim 1, wherein predicting the organizational improvement comprises predicting at least one of:
an optimal overall staffing level of a healthcare facility;
an optimal staffing level for a particular type of caregiver;
an optimal ratio of a first type of caregiver to a second type of caregiver; or
an amount of financing that will be required for a hierarchical category of the plurality of hierarchical categories.

10. The computer-implemented method of claim 1, wherein predicting the organizational improvement comprises predicting an optimal staffing level of a particular type of caregiver, the particular type of caregiver being one of:
a primary care physician (PCP);
an advanced practice provider (APP);
a registered nurse (RN);
a medical assistant (MA); or
a care manager (CM).

11. The computer-implemented method of claim 1, wherein predicting the organizational improvement comprises predicting an amount of financing that will be required for a service section, and the service section comprises at least one of:
ambulance and transportation;
ancillary services;
durable medical equipment (DME) and supplies;
emergency department (ED) care;
home based care;
hospital inpatient care;
hospital outpatient care;
institutional care;
lab and/or pathology;
office and/or clinic care;
treatment and evaluation of other sites;
physical, occupational, and/or speech therapy;
radiology;
retail prescriptions; or
specialty pharma.

12. The computer-implemented method of claim 1, wherein predicting the clinical operational improvement comprises predicting an optimal bundle of services for a patient of the plurality of patients by assigning the patient a bundle of services corresponding to a category classification of the patient.

13. The computer-implemented method of claim 1, wherein predicting the clinical operational improvement comprises predicting bundles of services for patients of the plurality of patients corresponding to category classifications of the patients, and wherein the bundles comprise: (i) types of touchpoints, (ii) time durations for the types of touchpoints, and (iii) numbers of times per bundle for the types of touchpoints.

14. The computer-implemented method of claim 1, wherein predicting the clinical operational improvement comprises modifying a care pathway and/or placing an intervention in the care pathway, the care pathway comprising a schedule of visits with healthcare providers.

15. A computer system for classifying medical patients, the computer system comprising one or more processors configured to:
receive respective medical data of a plurality of patients;
classify one or more patients of the plurality of patients into respective categories of a plurality of hierarchical categories based on the received respective medical data;
predict, based on the respective classifications of the patients into the respective categories, an organizational or clinical operational improvement; and
display, on a display, the predicted organizational or clinical operational improvement.

16. The computer system of claim 15, wherein the one or more processors are further configured to classify the patients by:
determining if a patient of the plurality of patients meets criteria of a first category of the plurality of hierarchical categories; and
if the patient does not meet the criteria of the first category, determining if the patient should be placed in a second category.

17. The computer system of claim 15, wherein the received respective medical data comprises at least one of:
a disease classification code, or information of a stay in a healthcare facility.

18. A computing device for classifying medical patients, the computing device comprising:
one or more processors; and
one or more memories;
the one or more memories having stored thereon computer-executable instructions that, when executed by the one or more processors, cause the computing device to:
receive respective medical data of a plurality of patients;
classify one or more patients of the plurality of patients into respective categories of a plurality of hierarchical categories based on the received respective medical data;
predict, based on the respective classifications of the patients into the respective categories, an organizational or clinical operational improvement; and
display, on a display, the predicted organizational or clinical operational improvement.

19. The computing device of claim 18, the one or more memories having stored thereon computer executable instructions that, when executed by the one or more processors, cause the computing device to classify the patients by:
determining if a patient of the plurality of patients meets criteria of a most severe health category of the plurality of hierarchical categories; and
if the patient does not meet the criteria of the most severe health category, determining if the patient should be placed in a second most severe health category.

20. The computing device of claim 18, the one or more memories having stored thereon computer executable instructions that, when executed by the one or more processors, cause the computing device to predict the organizational improvement by predicting an optimal staffing level for a particular type of caregiver.
